(19) United States
(12) Patent Application Publication
(10) Pub. No.: US 2008/0147192 A1
(43) Pub. Date: Jun. 19, 2008

(54) Percutaneous spinal implants and methods

(75) Inventors: Avram Allan Edidin, Sunnyvaly, CA (US); Hugues F. Malandain, Mountain View, CA (US)

Correspondence Address: FITZPATRICK CELLA (MEDTRONIC), 30 ROCKEFELLER PLAZA, NEW YORK, NY, 10112-3800, US

(73) Assignee: Kyphon, Inc., Sunnyvale, CA (US)

(21) Appl. No.: 11/788,752

(22) Filed: Apr. 19, 2007

Related U.S. Application Data
(63) Continuation of application No. 11/252,880, filed on Oct. 19, 2005.
Application No. 11/252,880 is a continuation-in-part of application No. 11/059,526, filed on Feb. 17, 2005.
(60) Provisional application No. 60/695,836, filed on Jul. 1, 2005.

(51) Int. Cl. A61F 2/44; A61M 29/00

(52) U.S. Cl. 623/17.16; 623/17.11; 623/17.12; 606/192

(57) ABSTRACT

An apparatus includes a guide shaft, an expansion member coupled to the guide shaft, and an actuator. The expansion member is configured to impart a force from within an interior of an implant to deform the implant. The actuator is coupled to the expansion member, the actuator is configured to move the expansion member from a first position to a second position.

### CROSS-REFERENCE TO RELATED APPLICATIONS

This application is a continuation-in-part of U.S. patent application Ser. No. 11/059,526, entitled “Apparatus and Method for Treatment of Spinal Conditions,” filed Feb. 17, 2005 and also claims the benefit of U.S. Provisional Application Ser. No. 60/695,836 entitled “Percutaneous Spinal Implants and Methods,” filed Jul. 1, 2005, each of which is incorporated herein by reference in its entirety.

### BACKGROUND

The invention relates generally to percutaneous spinal implants, and more particularly, to percutaneous spinal implants for implantation between adjacent spinous processes.

A back condition that impacts many individuals is spinal stenosis. Spinal stenosis is a progressive narrowing of the spinal canal that causes compression of the spinal cord. Each vertebra in the spinal column has an opening that extends through it. The openings are aligned vertically to form the spinal canal. The spinal cord runs through the spinal canal. As the spinal canal narrows, the spinal cord and nerve roots extending from the spinal cord and between adjacent vertebrae are compressed and may become inflamed. Spinal stenosis can cause pain, weakness, numbness, burning sensations, tingling, and in particularly severe cases, may cause loss of bladder or bowel function, or paralysis. The legs, calves and buttocks are most commonly affected by spinal stenosis, however, the shoulders and arms may also be affected.

Mild cases of spinal stenosis may be treated with rest or restricted activity, non-steroidal anti-inflammatory drugs (e.g., aspirin), corticosteroid injections (epidural steroids), and or physical therapy. Some patients find that bending forward, sitting or lying down may help relieve the pain. This may be due to bending forward creates more vertebral space, which may temporarily relieve nerve compression. Because spinal stenosis is a progressive disease, the source of pressure may have to be surgically corrected (decompressive laminectomy) as the patient has increasing pain. The surgical procedure can remove bone and other tissues that have impinged upon the spinal canal or put pressure on the spinal cord. Two adjacent vertebrae may also be fused during the surgical procedure to prevent an area of instability, improper alignment or slippage, such as that caused by spondylolisthesis. Surgical decompression can relieve pressure on the spinal cord or spinal nerve by widening the spinal canal to create more space. This procedure requires that the patient be given a general anesthesia as an incision is made in the patient to access the spine to remove the areas that are contributing to the pressure. This procedure, however, may result in blood loss and an increased chance of significant complications, and usually results in an extended hospital stay.

Minimally invasive procedures have been developed to provide access to the space between adjacent spinous processes such that major surgery is not required. Such known procedures, however, may not be suitable in conditions where the spinous processes are severely compressed. Moreover, such procedures typically involve large or multiple incisions.

Thus, a need exists for improvements in the treatment of spinal conditions such as spinal stenosis.

### SUMMARY OF THE INVENTION

An apparatus includes a guide shaft, an expansion member coupled to the guide shaft, and an actuator. The expansion member is configured to impart a force from within an interior of an implant to deform the implant. The actuator is coupled to the expansion member, the actuator is configured to move the expansion member from a first position to a second position.

An apparatus includes an elongate member having a proximal portion configured to be deformed from a first configuration to a second configuration under at least one of an axial load or a radial load. The elongate member has a distal portion configured to be deformed from a first configuration to a second configuration under at least one of an axial load or a radial load. A central portion is positioned between the proximal portion and the distal portion. The central portion is configured to engage adjacent spinous processes.

### BRIEF DESCRIPTION OF THE DRAWINGS

FIG. 1 is a schematic illustration of a posterior view of a medical device according to an embodiment of the invention in a first configuration adjacent two adjacent spinous processes.

FIG. 2 is a schematic illustration of a posterior view of a medical device according to an embodiment of the invention in a second configuration adjacent two adjacent spinous processes.

FIG. 3 is a schematic illustration of an expanding element according to an embodiment of the invention in a first configuration.

FIG. 4 is a schematic illustration of a side view of the deforming element illustrated in FIG. 3.

FIG. 5 is a side view of a medical device according to an embodiment of the invention in a first configuration.

FIG. 6 is a side view of the medical device illustrated in FIG. 5 in a second configuration.

FIG. 7 is a perspective view of a medical device according to an embodiment of the invention in a first configuration.

FIG. 8 is a posterior view of a medical device according to an embodiment of the invention, a portion of which is in a second configuration.

FIG. 9 is a posterior view of the medical device illustrated in FIG. 7 fully deployed in the second configuration.

FIG. 10 is a front plan view of the medical device illustrated in FIG. 7 in the second configuration.

FIG. 11 is a cross-sectional, side view of a medical device according to another embodiment of the invention in a first configuration.

FIG. 12 is a cross sectional, side view of the medical device illustrated in FIG. 11 in a partially expanded configuration.

FIG. 13 is a posterior view of the medical device illustrated in FIG. 11 inserted between adjacent spinous processes in a second configuration.

FIG. 14 is a lateral view of the medical device illustrated in FIG. 11 inserted between adjacent spinous processes in a second configuration.

FIG. 15 is a perspective view of an implant expansion device according to an embodiment of the invention in a first position.

FIG. 16 is a perspective view of the implant expansion device illustrated in FIG. 15 in a second position.

FIG. 17 is a partial cross-sectional illustration of the implant expansion device as illustrated in FIG. 15 inserted in a spinal implant.

FIG. 18 is a partial cross-sectional illustration of the implant expansion device as illustrated in FIG. 16 inserted in a spinal implant.

FIG. 19 is a side view of a partially expanded spinal implant.

FIG. 20 is a side view of an expanded spinal implant.

FIG. 21 is a cross-sectional, side view of an implant expansion device according to an alternative embodiment of the invention in a first configuration.

FIG. 22 is a cross-sectional, side view of the implant expansion device illustrated in FIG. 21 in a second configuration.

FIG. 23 is a cross-sectional, plan view of an implant expansion device according to a further embodiment of the invention in a first configuration.

FIG. 24 is a partial side view of an implant for use with the implant expansion device illustrated in FIG. 23.

FIG. 25 is a cross-sectional, plan view of the implant expansion device illustrated in FIG. 23 in a second configuration.

FIG. 26 is a cross-sectional, plan view of an implant expansion device according to another embodiment of the invention in a first configuration.

FIG. 27 is a cross-sectional, side view of the implant expansion device illustrated in FIG. 26.

FIGS. 28 and 29 illustrate a posterior view of a spinal implant expandable by an expansion device implant expander according to another embodiment of the invention in a first configuration and a second configuration, respectively.

FIG. 30 illustrates a cross-sectional, side view of a spinal implant according to an embodiment of the invention.

FIG. 31 is a cross-sectional, side view and FIG. 32 is a side view of an implant expansion device according to an embodiment of the invention for use with the spinal implant illustrated in FIG. 30.

FIGS. 33 and 34 illustrate the use of the implant expansion device illustrated in FIGS. 31 and 32 with the spinal implant illustrated in FIG. 30.

### DETAILED DESCRIPTION

An apparatus includes an elongate member having a proximal portion configured to be deformed from a first configuration to a second configuration under, for example, an axial load or a radial load. The elongate member has a distal portion configured to be deformed from a first configuration to a second configuration under, for example, an axial load or a radial load. A central portion is positioned between the proximal portion and the distal portion. The central portion is configured to engage adjacent spinous processes.

In some embodiments of the invention, the elongate member can have multiple portions that each move from a first configuration to a second configuration, either simultaneously or serially. Additionally, the device, or portions thereof, can be in many positions during the movement from the first configuration to the second configuration. For ease of reference, the entire device is referred to as being in either a first configuration or a second configuration.

FIG. 1 is a schematic illustration of a medical device according to an embodiment of the invention adjacent two adjacent spinous processes. The medical device 10 includes a proximal portion 12, a distal portion 14 and a central portion 16. The medical device 10 has a first configuration in which it can be inserted between adjacent spinous processes S. The central portion 16 is configured to contact the spinous processes S to prevent over-extension/compression of the spinous processes S. In some embodiments, the central portion 16 does not substantially distract the adjacent spinous processes S. In other embodiments, the central portion 16 does not distract the adjacent spinous processes S.

In the first configuration, the proximal portion 12, the distal portion 14 and the central portion 16 are coaxial (i.e., share a common longitudinal axis). In some embodiments, the proximal portion 12, the distal portion 14 and the central portion 16 define a tube having a constant inner diameter. In other embodiments, the proximal portion 12, the distal portion 14 and the central portion 16 define a tube having a constant outer diameter and/or inner diameter.

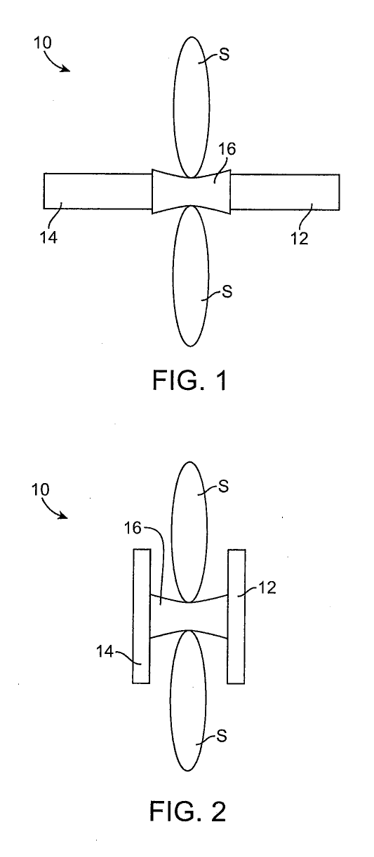

The medical device 10 can be moved from the first configuration to a second configuration as illustrated in FIG. 2. In the second configuration, the proximal portion 12 and the distal portion 14 are positioned to limit lateral movement of the device 10 with respect to the spinous processes S. The proximal portion 12 and the distal portion 14 are configured to engage the spinous process (i.e., either directly or through surrounding tissue) in the second configuration. For purposes of clarity, the tissue surrounding the spinous processes S is not illustrated.

In some embodiments, the proximal portion 12, the distal portion 14 and the central portion 16 are monolithically formed. In other embodiments, one or more of the proximal portion 12, the distal portion 14 and the central portion 16 are separate components that can be coupled together to form the medical device 10. For example, the proximal portion 12 and distal portion 14 can be monolithically formed and the central portion can be a separate component that is coupled thereto.

In use, the spinous processes S can be distracted prior to inserting the medical device 10. Distraction of spinous processes is disclosed, for example, in U.S. application Ser. No. 11/059,526, incorporated herein by reference in its entirety. When the spinous processes are distracted, a trocar can be used to define an access passage for the medical device 10. In some embodiments, the trocar can be used to define the passage as well as distract the spinous processes S. Once an access passage is defined, the medical device 10 is inserted percutaneously and advanced between the spinous processes, distal end 14 first, until the central portion 16 is located between the spinous processes S. Once the medical device 10 is in place between the spinous processes, the proximal portion 12 and the distal portion 14 are moved to the second configuration, either serially or simultaneously.

In some embodiments, the medical device 10 is inserted percutaneously (i.e., through an opening in the skin) and in a minimally invasive manner. For example, as discussed in detail herein, the size of portions of the implant is expanded after the implant is inserted between the spinous processes. Once expanded, the size of the expanded portions of the implant is greater than the size of the opening. For example, the size of the opening/incision in the skin may be between 3 millimeters in length and 25 millimeters in length. In some embodiments, the size of the implant in the expanded configuration is between 3 and 25 millimeters.

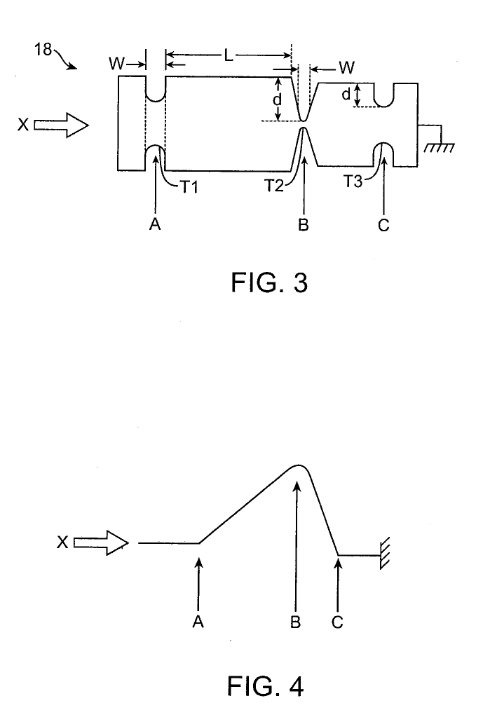

FIG. 3 is a schematic illustration of a deformable element 18 that is representative of the characteristics of, for example, the distal portion 14 of the medical device 10 in a first configuration. The deformable member 18 includes cutouts A, B, C along its length to define weak points that allow the deformable member 18 to deform in a predetermined manner. Depending upon the depth d of the cutouts A, B, C and the width w of the throats T1, T2, T3, the manner in which the deformable member 18 deforms under an applied load can be controlled and varied. Additionally, depending upon the length L between the cutouts A, B, C (i.e., the length of the material between the cutouts) the manner in which the deformable member 18 deforms can be controlled and varied.

FIG. 4 is a schematic illustration of the expansion properties of the deformable member 18 illustrated in FIG. 3. When a load is applied, for example, in the direction indicated by arrow X, the deformable member 18 deforms in a predetermined manner based on the characteristics of the deformable member 18 as described above. As illustrated in FIG. 4, the deformable member 18 deforms most at cutouts B and C due to the configuration of the cutout C and the short distance between cutouts B and C. In some embodiments, the length of the deformable member 18 between cutouts B and C is sized to fit adjacent a spinous process.

The deformable member 18 is stiffer at cutout A due to the shallow depth of cutout A. As indicated in FIG. 4, a smooth transition is defined by the deformable member 18 between cutouts A and B. Such a smooth transition causes less stress on the tissue surrounding a spinous process than a more drastic transition such as between cutouts B and C. The dimensions and configuration of the deformable member 18 can also determine the timing of the deformation at the various cutouts. The weaker (i.e., deeper and wider) cutouts deform before the stronger (i.e., shallower and narrower) cutouts.

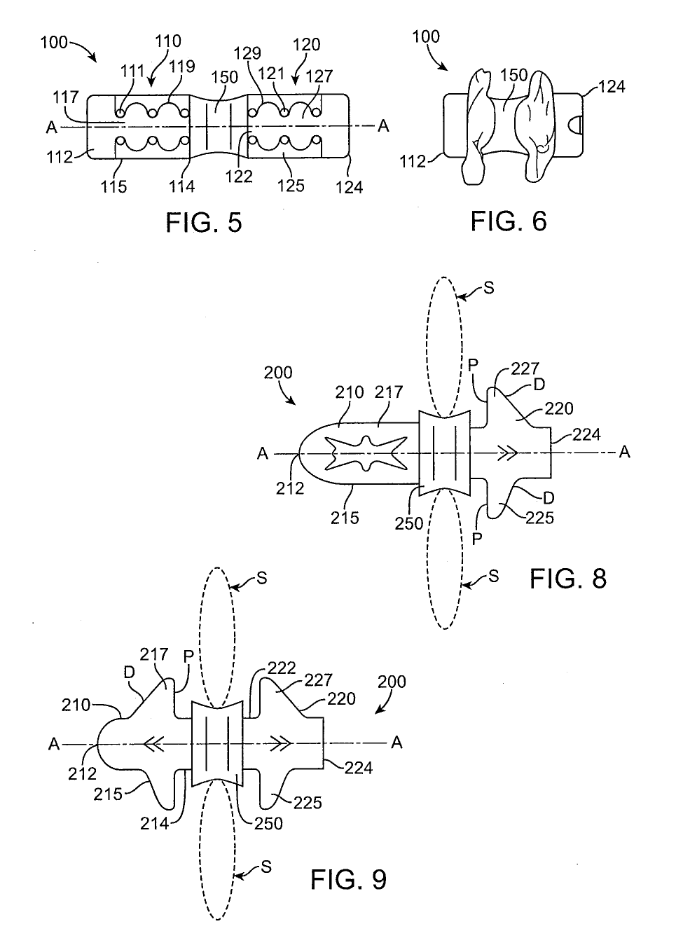

FIGS. 5 and 6 illustrate a spinal implant 100 in a first configuration and second configuration, respectively. As shown in FIG. 5, the spinal implant 100 is collapsed in a first configuration and can be inserted between adjacent spinous processes. The spinal implant 100 has a first expandable portion 110, a second expandable portion 120 and a central portion 150. The first expandable portion 110 has a first end 112 and a second end 114. The second expandable portion 120 has a first end 122 and a second end 124. The central portion 150 is coupled between second end 114 and first end 122. In some embodiment, the spinal implant 100 is monolithically formed.

The first expandable portion 110, the second expandable portion 120 and the central portion 150 have a common longitudinal axis A along the length of spinal implant 100. The central portion 150 can have the same inner diameter as first expandable portion 110 and the second expandable portion 120. In some embodiments, the outer diameter of the central portion 150 is smaller than the outer diameter of the first expandable portion 110 and the second expandable portion 120.

In use, spinal implant 100 is inserted percutaneously between adjacent spinous processes. The first expandable portion 110 is inserted first and is moved past the spinous processes until the central portion 150 is positioned between the spinous processes. The outer diameter of the central portion 150 can be slightly smaller than the space between the spinous processes to account for surrounding ligaments and tissue. In some embodiments, the central portion directly contacts the spinous processes between which it is positioned. In some embodiments, the central portion of spinal implant 100 is a fixed size and is not compressible or expandable.

The first expandable portion 110 includes expanding members 115, 117 and 119. Between the expanding members 115, 117, 119, openings 111 are defined. As discussed above, the size and shape of the openings 111 influence the manner in which the expanding members 115, 117, 119 deform when an axial load is applied. The second expandable portion 120 includes expanding members 125, 127 and 129. Between the expanding members 125, 127, 129, openings 121 are defined. As discussed above, the size and shape of the openings 121 influence the manner in which the expanding members 125, 127, 129 deform when an axial load is applied.

When an axial load is applied to the spinal implant 100, the spinal implant 100 expands to a second configuration as illustrated in FIG. 6. In the second configuration, first end 112 and second end 114 of the first expandable portion 110 move towards each other and expanding members 115, 117, 119 project substantially laterally away from the longitudinal axis A. Likewise, first end 122 and second end 124 of the second expandable portion 120 move towards one another and expanding members 125, 127, 129 project laterally away from the longitudinal axis A. The expanding members 115, 117, 119, 125, 127, 129 in the second configuration form projections that extend to positions adjacent to the spinous processes between which the spinal implant 100 is inserted. In the second configuration, the expanding members 115, 117, 119, 125, 127, 129 inhibit lateral movement of the spinal implant 100, while the central portion 150 prevents the adjacent spinous processes from moving together any closer than the distance defined by the diameter of the central portion 150.

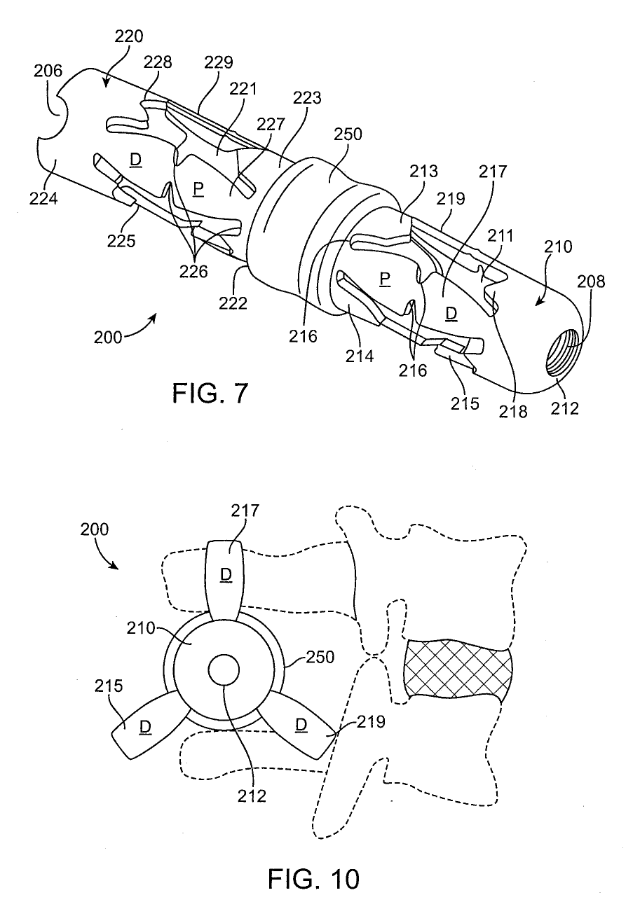

A spinal implant 200 according to an embodiment of the invention is illustrated in FIGS. 7-9 in various configurations. Spinal implant 200 is illustrated in a completely collapsed configuration in FIG. 7 and can be inserted between adjacent spinous processes. The spinal implant 200 has a first expandable portion 210, a second expandable portion 220 and a central portion 250. The first expandable portion 210 has a first end 212 and a second end 214. The second expandable portion 220 has a first end 222 and a second end 224. The central portion 250 is coupled between second end 214 and first end 222.

The first expandable portion 210, the second expandable portion 220 and the central portion 250 have a common longitudinal axis A along the length of spinal implant 200. The central portion 250 can have the same inner diameter as first expandable portion 210 and the second expandable portion 220. The outer diameter of the central portion 250 is greater than the outer diameter of the first expandable portion 210 and the second expandable portion 220. The central portion 250 can be monolithically formed with the first expandable portion 210 and the second expandable portion 220 or can be a separately formed sleeve coupled thereto or thereupon.

In use, spinal implant 200 is inserted percutaneously between adjacent spinous processes S. The first expandable portion 210 is inserted first and is moved past the spinous processes S until the central portion 250 is positioned between the spinous processes S. The outer diameter of the central portion 250 can be slightly smaller than the space between the spinous processes S to account for surrounding ligaments and tissue. In some embodiments, the central portion 250 directly contacts the spinous processes S between which it is positioned. In some embodiments, the central portion 250 of spinal implant 200 is a fixed size and is not compressible or expandable. In other embodiments, the central portion 250 can compress to conform to the shape of the spinous processes.

The first expandable portion 210 includes expanding members 215, 217 and 219. Between the expanding members 215, 217, 219, openings 211 are defined. As discussed above, the size and shape of the openings 211 influence the manner in which the expanding members 215, 217, 219 deform when an axial load is applied. Each expanding member 215, 217, 219 of the first expandable portion 210 includes a tab 213 extending into the opening 211 and an opposing mating slot 218. In some embodiments, the first end 212 of the first expandable portion 210 is rounded to facilitate insertion of the spinal implant 200.

The second expandable portion 220 includes expanding members 225, 227 and 229. Between the expanding members 225, 227, 229, openings 221 are defined. As discussed above, the size and shape of the openings 221 influence the manner in which the expanding members 225, 227, 229 deform when an axial load is applied. Each expanding member 225, 227, 229 of the second expandable portion 220 includes a tab 223 extending into the opening 221 and an opposing mating slot 228.

When an axial load is applied to the spinal implant 200, the spinal implant moves to a partially expanded configuration as illustrated in FIG. 8. In the partially expanded configuration, first end 222 and second end 224 of the second expandable portion 220 move towards one another and expanding members 225, 227, 229 project laterally away from the longitudinal axis A. To prevent the second expandable portion 220 from over-expanding, the tab 223 engages slot 228 and acts as a positive stop. As the axial load continues to be imparted to the spinal implant 200 after the tab 223 engages slot 228, the load is transferred to the first expandable portion 210. Accordingly, the first end 212 and the second end 214 then move towards one another until tab 213 engages slot 218 in the fully expanded configuration illustrated in FIG. 9. In the second configuration, expanding members 215, 217, 219 project laterally away from the longitudinal axis A. In some alternative embodiments, the first expandable portion and the second expandable portion expand simultaneously under an axial load.

The order of expansion of the spinal implant 200 can be controlled by varying the size of openings 211 and 221. For example, in the embodiments shown in FIGS. 7-9, the opening 221 is slightly larger than the opening 211. Accordingly, the notches 226 are slightly larger than the notches 216. As discussed above with respect to FIGS. 3 and 4, for this reason, the second expandable portion 220 will expand before the first expandable portion 210 under an axial load.

In the second configuration, the expanding members 215, 217, 219, 225, 227, 229 form projections that extend adjacent the spinous processes S. Once in the second configuration, the expanding members 215, 217, 219, 225, 227, 229 inhibit lateral movement of the spinal implant 200, while the central portion 250 prevents the adjacent spinous processes from moving together any closer than the distance defined by the diameter of the central portion 250.

The portion P of each of the expanding members 215, 217, 219, 225, 227, 229 proximal to the spinous process S expands such that portion P is substantially parallel to the spinous process S. The portion D of each of the expanding members 215, 217, 219, 225, 227, 229 distal from the spinous process S is angled such that less tension is imparted to the surrounding tissue.

In the second configuration, the expanding members 225, 227, 229 are separate by approximately 120 degrees from an axial view as illustrated in FIG. 10. While three expanding members are illustrated, two or more expanding members may be used and arranged in an overlapping or interleaved fashion when multiple implants 200 are inserted between multiple adjacent spinous processes. Additionally, regardless of the number of expanding members provided, the adjacent expanding members need not be separated by equal angles or distances.

The spinal implant 200 is deformed by a compressive force imparted substantially along the longitudinal axis A of the spinal implant 200. The compressive force is imparted, for example, by attaching a rod (not illustrated) to the first end 212 of the first expandable portion 210 and drawing the rod along the longitudinal axis while imparting an opposing force against the second end 224 of the second expandable portion 220. The opposing forces result in a compressive force causing the spinal implant 200 to expand as discussed above.

The rod used to impart compressive force to the spinal implant 200 can be removably coupled to the spinal implant 200. For example, the spinal implant 200 can include threads 208 at the first end 212 of the first expandable portion 210. The force opposing that imparted by the rod can be applied by using a push bar (not illustrated) that is removably coupled to the second end 224 of the second expandable portion 220. The push rod can be aligned with the spinal implant 200 by an alignment notch 206 at the second end 224. The spinal implant 200 can also be deformed in a variety of other ways, examples of which are discussed in detail below.

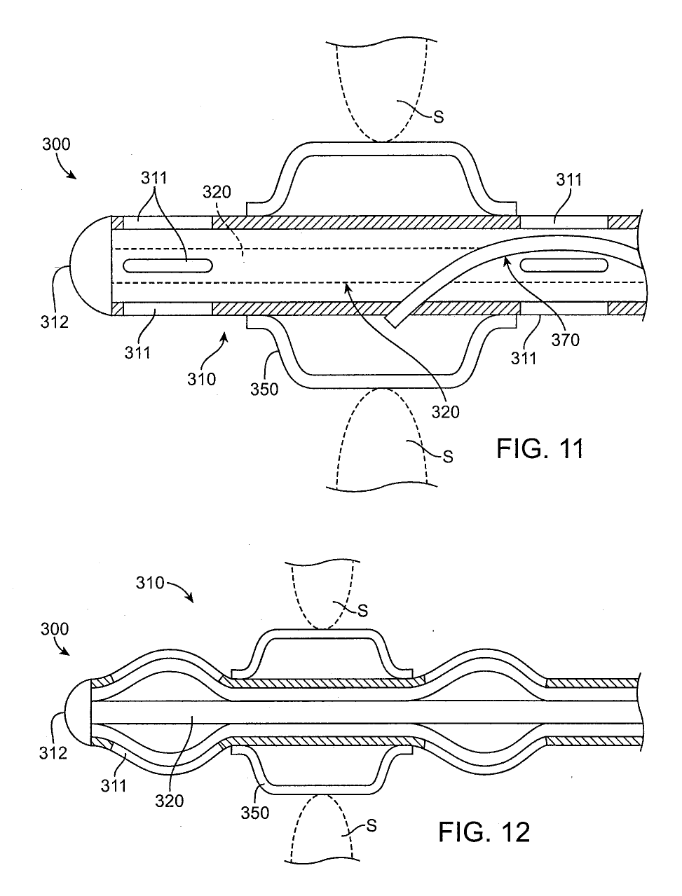

FIGS. 11-14 illustrate a spinal implant 300 according to an embodiment of the invention. Spinal implant 300 includes an elongated tube 310 configured to be positioned between adjacent spinous processes S and having a first end 312 and a second end 314. The elongated tube 310 has longitudinal slots 311 defined along its length at predetermined locations. The slots 311 are configured to allow portions of the elongated tube 310 to expand outwardly to form projections 317. An inflatable member 350 is disposed about the elongated tube between adjacent sets of slots 311.

The inflatable member 350 is configured to be positioned between adjacent spinous processes S as illustrated in FIGS. 11-14. Once inserted between the adjacent spinous processes, the inflatable member 350 is inflated with a liquid and/or a gas, which can be, for example, a biocompatible material. The inflatable member 350 is inflated to maintain the spinal implant 300 in position between the spinous processes S. In some embodiments, the inflatable member 350 is configured to at least partially distract the spinous processes S when inflated. The inflatable member 350 can be inflated to varied dimensions to account for different spacing between spinous processes S.

The inflatable member 350 can be inflated via an inflation tube 370 inserted through the spinal implant 300 once spinal implant 300 is in position between the spinous processes S. Either before or after the inflatable member 350 is inflated, the projections 317 are expanded. To expand the projections 317, an axial force is applied to the spinal implant 300 using draw bar 320, which is coupled to the first end 312 of the spinal implant 300.

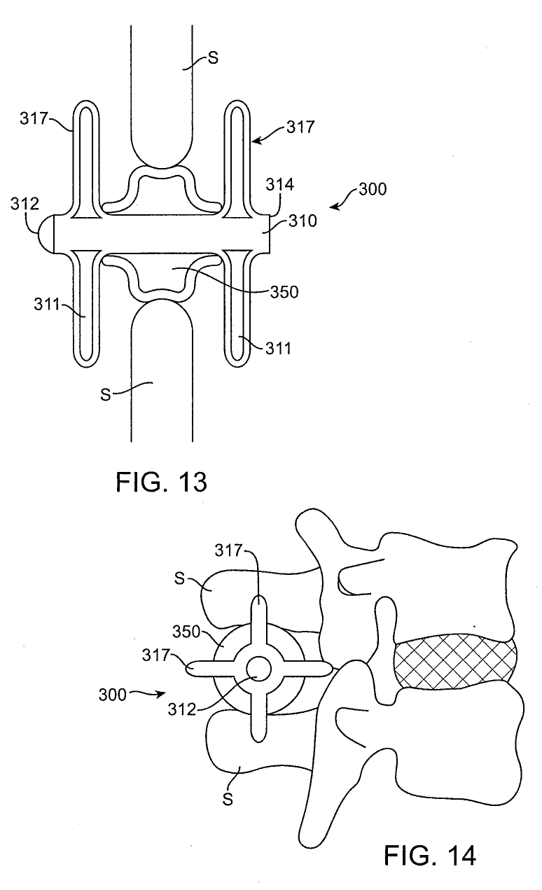

As the draw bar 320 is pulled, the axial load causes the projections 317 to buckle outwardly, thereby preventing the spinal implant from lateral movement with respect to the spinous processes S. FIG. 12 is an illustration of the spinal implant 300 during deformation, the projections 317 being only partially formed. Although illustrated as deforming simultaneously, the slots 311 alternatively can be dimensioned such that the deformation occurs at different times as described above. Once the spinal implant is in the expanded configuration (see FIG. 13), the draw bar 320 is removed from the elongated tube 310.

The orientation of the spinal implant 300 need not be such that two projections are substantially parallel to the axis of the portion of the spine to which they are adjacent as illustrated in FIG. 14. For example, the spinal implant 300 can be oriented such that each of the projections 317 is at a 45 degree angle with respect to the spinal axis.

The spinal implants 100, 200, 300 can be deformed from their first configuration to their second configuration using a variety of expansion devices. For example, portions of the spinal implants 100, 200, 300, as well as other types of implants I, can be deformed using expansion devices described below. While various types of implants I are illustrated, the various expansion devices described can be used with any of the implants described herein.

FIG. 15 illustrates a portion of expansion device 400 in a collapsed configuration. Expansion device 400 can be used to selectively form protrusions on the implant I (not illustrated in FIG. 15) at desired locations. The expansion device 400 includes a guide shaft 410, which can guide the expansion device 400 into the implant I and a cam actuator 450 mounted thereto and positionable into an eccentric position. The expansion device 400 has a longitudinal axis A and the cam actuator 450 has a cam axis C that is laterally offset from the longitudinal axis A by a distance d. FIG. 16 illustrates the expansion device 400 in the expanded configuration with the cam actuator 450 having been rotated about the cam axis C.

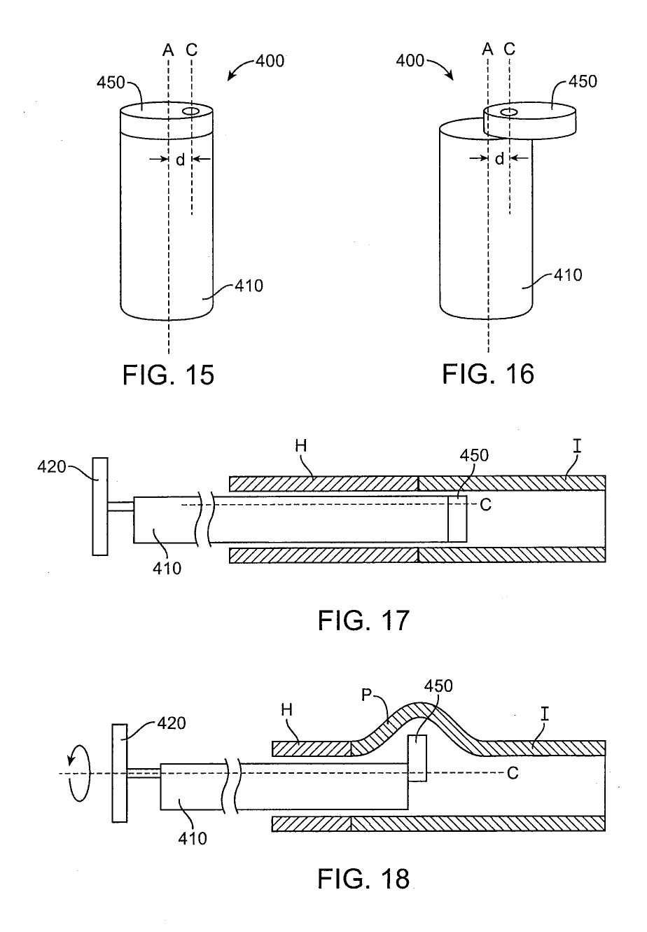

The expansion device 400 can be inserted into an implant I through an implant holder H as illustrated in FIG. 17. The implant holder H is coupled to the implant and is configured to hold the implant in position while the expansion device 400 is being manipulated to deform the implant I. Once the implant I is satisfactorily deformed, the implant holder H can be detached from the implant I and removed from the patient, leaving the implant I behind.

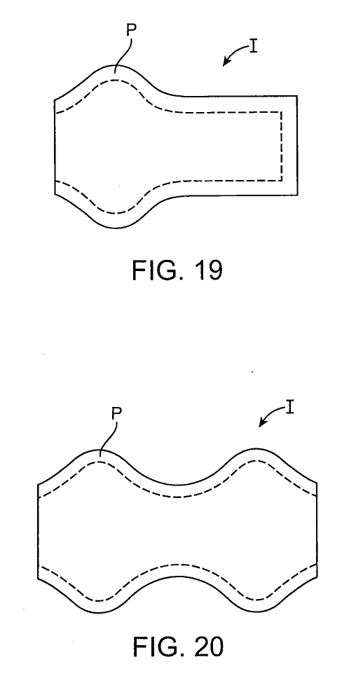

Referring to FIGS. 17 and 18, the expansion device 400 includes a handle 420 that is used to deploy the cam actuator 450. When the handle 420 is rotated, the cam actuator 450 is deployed and deforms the implant I. Once the cam actuator 450 is fully deployed (e.g., 180 degrees from its original position) and locked in place, the entire expansion device 400 is rotated to deform the implant I around the circumference of implant I. The cam actuator 450 circumscribes a locus of points that is outside the original diameter of the implant I, forming the projection P (see FIG. 19). The expansion device 400 can be rotated either by grasping the guide shaft 410 or by using the handle 420 after it has been locked in place.

The expansion device 400 can be used to form multiple projections P. Once a first projection P is formed, the cam actuator 450 can be rotated back to its first configuration and the expansion device 400 advanced through the implant I to a second position. When the expansion device 400 is appropriately positioned, the cam actuator 450 can again be deployed and the expansion device 400 rotated to form a second projection P (see FIG. 20). In some embodiments, the implant I is positioned between adjacent spinous processes and the projections P are formed on the sides of the spinous processes to prevent lateral (i.e., axial) displacement of the implant I.

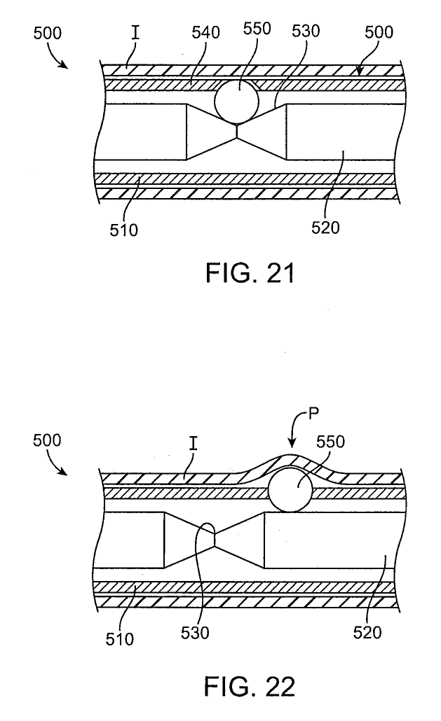

An alternative expansion device 500 is illustrated in FIGS. 21 and 22. FIG. 21 illustrates the expansion device 500 in a first configuration and FIG. 22 illustrates the expansion device 500 in a second configuration. The expansion device 500 includes a guide shaft 510 that is inserted into an implant I. An axial cam shaft actuator 520 is slidably disposed within the guide shaft 520. The axial cam shaft actuator 520 has a sloped recess 530 to receive a movable object 550. When the cam shaft actuator 520 is moved, the movable object 550 is displaced along the sloped recess 530 until it protrudes through an opening 540 in the guide shaft 510.

The movable object 550 is configured to displace a portion of the implant I, thereby forming a projection P. Multiple movable objects 550 can be used around the circumference of the guide shaft 510 to form a radially extending protrusions P around the circumference of the implant I. Additionally, the protrusions can be formed at multiple locations along the length of the implant I by advancing the expansion device 500 along the length of the implant to a second position as discussed above. Alternatively, the expansion device can have multiple recesses that displace other sets of movable objects.

In alternative embodiments, the expansion device can also serve as an implant. For example, the expansion device 500 can be inserted between adjacent spinous processes S, the movable objects moved out through openings 540, and the expansion device 500 left behind in the body. In such an embodiment, the movable objects prevent the expansion device 506 from lateral movement with respect to the spinous processes S.

In another alternative embodiment, rather than having openings 540 in the expansion device 500, the movable objects 550 can be positioned against a weaker (e.g., thinner) portion of the wall of the expansion device and move that portion of the expansion device 500 to a protruded configuration.

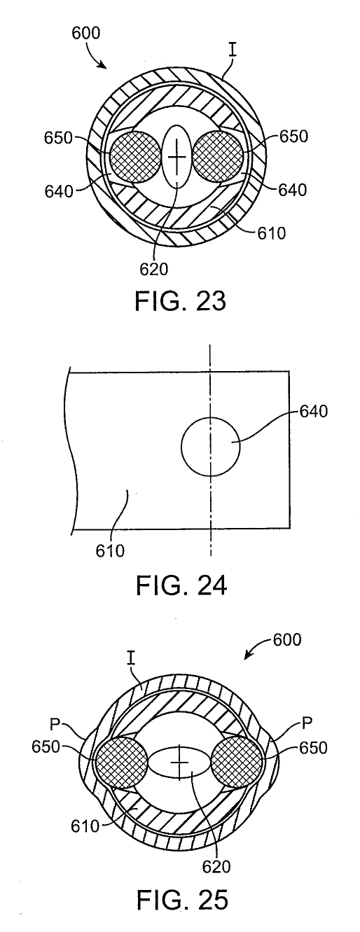

Another alternative expansion device 600 is illustrated in FIGS. 23-25. FIG. 23 illustrates the expansion device 600 in a first configuration and FIG. 25 illustrates the expansion device in a second configuration. The expansion device 600 includes a guide shaft 610 that is inserted into an implant I. The guide shaft 610 has openings 640 defined therein. An axial cam shaft actuator 620 is rotatably coupled within the guide shaft 610. Displaceable objects 650 are positioned within the guide shaft 610 and are configured to protrude through the openings 640 in the guide shaft 610. When the cam shaft actuator 620 is rotated approximately 90 degrees, the movable objects 650 move through the openings 640 and deform the implant I, forming the projection P. Alternatively, the expansion device can have multiple cams that displace other sets of movable objects.

Multiple movable objects 650 can be used around the circumference of the guide shaft 610 to form radially extending protrusions P around the implant I. Additionally, the protrusions can be formed at multiple locations along the length of the implant I by advancing the expansion device 600 along the length of the implant I to a second position as discussed above.

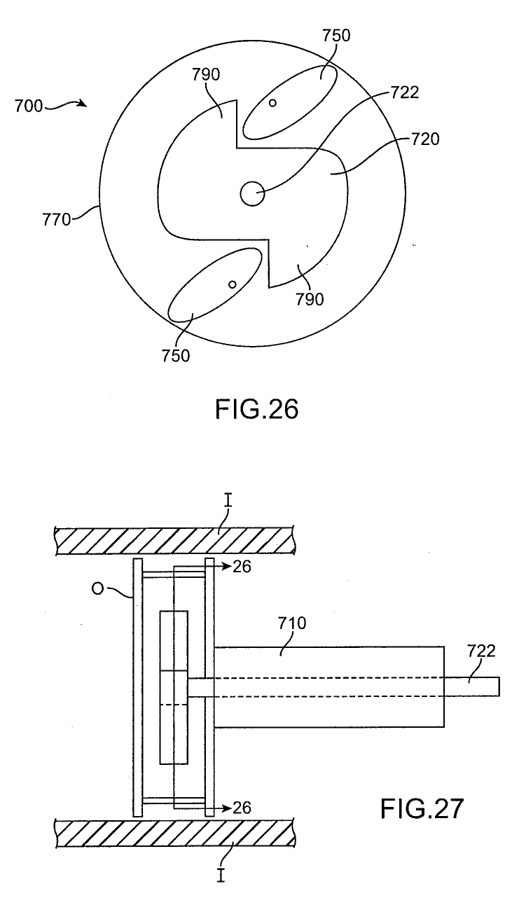

An implant expansion device 700 is illustrated in FIGS. 26 and 27. The implant expansion device 700 is configured to be inserted into an implant I. The implant 700 includes a guide shaft 710 coupled to a housing 770. A cam actuator 720 is rotatably mounted within the housing 770 and includes arms 790 that extend in opposite directions from one another. The cam actuator 720 is rotated using rod 722.

As the cam actuator 720 rotates, the arms 790 engage movable objects 750. The movable objects 750 are configured to project out of the housing 770 when the cam actuator is rotated in a clockwise manner. Once the movable objects 750 are fully extended, they engage the implant I and the expansion device 700 can be rotated a complete revolution to form a protrusion in the implant I.

After one protrusion is formed, the rod 722 can be rotated counterclockwise to disengage the movable objects 750 from the implant I. Once disengaged, the expansion device 700 can be advanced to another location within the implant I as discussed above.

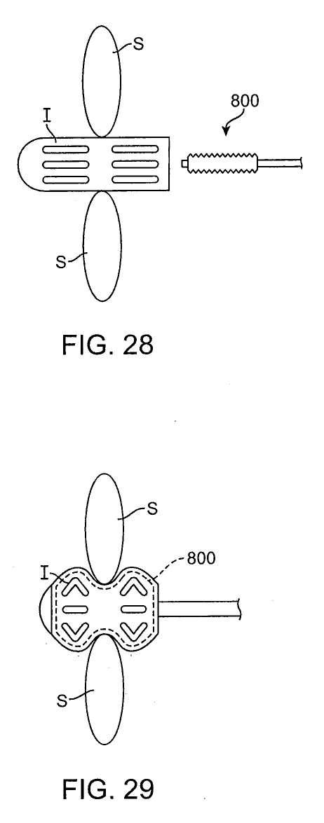

In some other embodiments, the implant I can be balloon actuated. FIG. 28 illustrates an implant I positioned between adjacent spinous processes S. A balloon actuator 800 in inserted into the implant I and expanded as illustrated in FIG. 29 to move the implant I to its expanded configuration. Once expanded, the balloon actuator 800 can be deflated and removed, leaving the implant I in an expanded configuration.

In some embodiments, the balloon actuator 800 can have multiple lobes, one that expands on each side of the spinous process S. In other embodiments, multiple balloon actuators 800 can be used to expand the implant I.

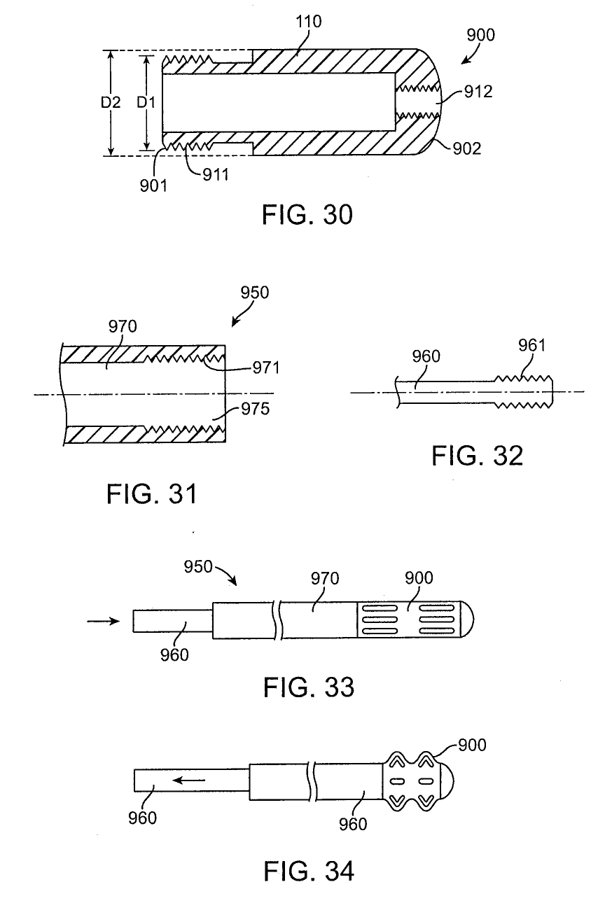

FIG. 30 is a cross-sectional view of an expandable implant 900 that can be expanded using an expansion device 950, illustrated in FIGS. 31-34. The implant 900 has an elongated body portion 910 having a first end 901 and a second end 902. The first end 901 has an externally threaded portion 911 and the second end 902 has an internally threaded portion 912. The implant 900 has a first outer diameter D1 at the externally threaded portion 911 and a second outer diameter D2, which wider than the first outer diameter D1.

The expansion device 950 includes a draw bar 960 and a compression bar 970. In some embodiments, the compression bar 970 defines a channel 975 having internal threads 971 to mate with the externally threaded portion 911 of the implant 900 (see FIG. 31). The draw bar 960 has external threads 961 to mate with the internally threaded portion 912 of implant 900.

In use, the compression bar 970 is coupled to the first end 901 of the implant 900 and abuts the implant 900 at the transition between the first outer diameter D1 and the second outer diameter D2, which serves as a stop for the compression bar 970. In some embodiments, the outer diameter of the entire implant 900 is substantially constant and the inner diameter of the compression bar 970 narrows to serve as the stop for the compression bar 970. With the compression bar 970 in place, the draw bar 960 is inserted through the channel 975 and is coupled to the second end 902 of the implant 900 via the internally threaded portion 912 of implant 900 (see FIG. 32). Once the compression bar 970 and the draw bar 960 are coupled to the implant 900, the draw bar 960 can be pulled while imparting an opposing force on the compression bar 970 to expand the implant 900 (see FIG. 33). When the implant 900 is fully expanded, the compression bar 970 and the draw bar 960 are removed and the implant is left behind the body.

With the expansion devices described herein, the location of protrusions can be selected in vivo, rather than having predetermined expansion locations. Such a configuration reduces the need to have multiple sizes of spacers available. Additionally, the timing of the deployment of the protrusions can be varied.

The various implants 100, 200, 300 described herein can be made from, for example, stainless steel, plastic, polyetheretherketone (PEEK), carbon fiber, ultra-high molecular weight (UHMW) polyethylene, etc. The material can have a tensile strength similar to or higher than that of bone.

### CONCLUSION

While various embodiments have been described above, it should be understood that they have been presented by way of example only, and not limitation. While embodiments have been particularly shown and described, it will be understood by those skilled in art that various changes in form and details may be made therein.

For example, although the embodiments above are primarily described as being spinal implants configured to be positioned between adjacent spinous processes, in alternative embodiments, the implants are configured to be positioned adjacent any bone, tissue or other bodily structure where it is desirable to maintain spacing while preventing axial or longitudinal movement of the implant.

While the implants described herein were primarily described as not distracting adjacent spinous processes, in alternative embodiments, the implants can be configured to expand to distract adjacent spinous processes.

Although described as being inserted directly between adjacent spinous processes, in alternative embodiments, the implants described above can be delivered through a cannula.

1.-21. (canceled)

22. An interspinous device for stabilizing at least one spinal motion segment comprising a first vertebra having a first spinous process and a second vertebra having a second spinous process, the device comprising:
an expandable member having an unexpanded configuration and an expanded configuration, wherein the expandable member in an expanded configuration has a size and shape configured for positioning between and providing distraction of the first and second spinous processes.

23. The device of claim 22, wherein the expandable member is a non-compliant balloon.

24. The device of claim 22, wherein the expandable member is a compliant balloon.

25. The device of claim 22, wherein the expandable member comprises a mesh material.

26. The device of claim 25, wherein the mesh material is coated with a non-porous material.

27. The device of claim 22, wherein the expandable member has a substantially H-shaped configuration.

28. The device of claim 22, wherein the expandable member comprises polyurethane.

29. The device of claim 22, further comprising at least one tab for anchoring the expandable member to the spinous processes.

30. The device of claim 29, further comprising a tether attached to the at least one tab.

31. The device of claim 22, further comprising at least one tether attached to the expandable member.

32. The device of claim 22, wherein the expandable member has a port for coupling to a source of an expansion medium.

33. The device of claim 22, wherein the port comprises a one-way valve mechanism.

34. The device of claim 22, wherein the expandable member is inflatable.

35. The device of claim 22, further comprising an expansion medium contained within the interior of the expandable member when in an expanded configuration.

36. The device of claim 22, further comprising at least one marker on a surface of the expandable member.

37. A system for stabilizing at least one spinal motion segment comprising a first vertebra having a first spinous process and a second vertebra having a second spinous process, the system comprising:
an expandable member having an unexpanded configuration and an expanded configuration, wherein the expandable member in an expanded configuration has a size and shape configured for positioning between and providing distraction of the first and second spinous processes, wherein the expandable member has a port; and an expansion medium fillable within the interior of the expandable member via the port.

38. The system of claim 37, further comprising at least one anchor for securing the expandable member to the spinal motion segment.

39. The system of claim 38, wherein the at least one anchor is a screw configured for securing the expandable member to a spinous process.

40. The system of claim 37, wherein the expansion medium comprises polyurethane.

41. A kit for stabilizing at least one spinal motion segment comprising a first vertebra having a first spinous process and a second vertebra having a second spinous process wherein a spinous ligament extends between the first and second spinous processes, the kit comprising:
at least one expandable member having an unexpanded configuration and an expanded configuration, wherein the at least one expandable member in an expanded configuration has a size and shape configured for positioning between and providing distraction of the first and second spinous processes; and
a cannula configured for percutaneous delivery to a target site within the spinal motion segment wherein the expandable member is deliverable through the cannula when in an unexpanded configuration.

42. The kit of claim 41, further comprising instructions for implanting the expandable member between the first and second spinous processes.

43. A method for stabilizing at least one spinal motion segment comprising a superior vertebra and an inferior vertebra, the method comprising:
providing the device of claim 21; forming an opening in the spinous ligament;
positioning the expandable member within the opening wherein the expandable member is in the unexpanded configuration;
and expanding the expandable member to the expanded configuration wherein the first and second vertebrae are distracted a selected distance.

44. The method of claim 43, wherein the expanding comprises inflating the expandable member with air.

45. The method of claim 44, the expanding further comprises filling the expandable member with a flowable medium.

46. The method of claim 43, wherein the steps of forming, positioning and expanding are performed through a percutaneous penetration in the patient's skin.

47. The method of claim 46, wherein the steps of forming, positioning and expanding are performed through a cannula positioned within the percutaneous penetration.

48. The method of claim 43, further comprising anchoring the expandable member to one or both of the spinous processes.

49. The method of claim 43, wherein the expandable member has a volume in the expanded configuration, the method further comprising adjusting the volume of the expandable member.

50. The method of claim 49, further comprising reducing the volume of the expandable member and removing the expandable member subsequent to reducing the volume.

51. A method for stabilizing at least one spinal motion segment comprising a superior vertebra and an inferior vertebra, the method comprising:
forming an opening in the spinous ligament;
positioning an expandable member within the opening wherein the expandable member is in an unexpanded configuration;
and selectively expanding the expandable member to an expanded configuration wherein the first and second vertebrae are distracted a selected distance.

52. An interspinous device for stabilizing at least one spinal motion segment comprising a first vertebra having a first spinous process and a second vertebra having a second spinous process, the device comprising:
an expandable member having an unexpanded configuration and an expanded configuration, wherein the expandable member in the unexpanded configuration has a size configured for positioning between the first and second spinous process and in the expanded configuration provides distraction of the first and second spinous processes, wherein the expandable member is mechanically actuatable from the unexpanded configuration to the expanded configuration.

53. The device of claim 52, wherein the expandable member comprises a plurality of parallel struts.

54. The device of claim 53, wherein the plurality of struts are affixed between two opposing hubs.

55. The device of claim 52, wherein the expandable member comprises a coiled band.

56. The device of claim 55, wherein the coiled band comprises inner and outer ends which are connectable to each other.

57. The device of claim 52, wherein the expandable member comprises a plurality of nested portions.

58. The device of claim 52, wherein the expandable member has a spherical configuration.

59. The device of claim 52, wherein the expandable member has an elliptical configuration.

60. The device of claim 52, wherein the expandable member has a disk configuration.

61. The device of claim 52, wherein the expandable member is self-expanding.

62. A system for stabilizing at least one spinal motion segment comprising a first vertebra having a first spinous process and a second vertebra having a second spinous process, the system comprising:
an expandable member having an unexpanded configuration and an expanded configuration, wherein the expandable member in the unexpanded configuration has a size configured for positioning between the first and second spinous process and in the expanded configuration provides distraction of the first and second spinous processes, wherein the expandable member is mechanically actuatable from the unexpanded configuration to the expanded configuration;
and means for mechanically actuating the expandable member.

63. The system of claim 62, wherein said mechanical actuation means is a guide wire.

64. A method for stabilizing a vertebrae relative to another vertebrae, the method comprising:
introducing an expandable interspinous device to laterally of an interspinous space of the vertebrae;
expanding the interspinous device;
and inserting the interspinous device within the interspinous space.

65. The method of claim 64, further comprising: distracting the interspinous space prior to inserting the interspinous device.

66. The method of claim 65, wherein the distracting comprises introducing a device within the interspinous space and expanding the distracting device.

67. The method of claim 66, wherein the distracting device is a balloon.

68. The method of claim 66, wherein the distracting device is a mechanical device.

69. The method of claim 67, wherein the distracting device is introduced on one side of the interspinous space and the expandable interspinous device is introduced on the opposite side of the interspinous space.

70. The method of claim 67, wherein the distracting device and the expandable interspinous device are introduced on the same side of the interspinous space.

71. A kit for stabilizing at least one spinal motion segment comprising a first vertebra having a first spinous process and a second vertebra having a second spinous process, the kit comprising: the interspinous device of claim 52; and a cannula configured for percutaneous delivery to a target site within the spinal motion segment wherein the expandable member is deliverable through the cannula when in an unexpanded configuration.

72. The kit of claim 71, further comprising instructions for implanting the expandable member between the first and second spinous processes.

73. A method for stabilizing at least one spinal motion segment comprising a superior vertebra and an inferior vertebra, the method comprising:
providing the device of claim 52;
forming an opening in the spinous ligament;
positioning the expandable member within the opening wherein the expandable member is in the unexpanded configuration; and
expanding the expandable member to the expanded configuration wherein the first and second vertebrae are distracted a selected distance.

74. The method of claim 73, wherein the expanding comprises mechanical actuation.

75. The method of claim 73, wherein the steps of forming, positioning and expanding are performed through a percutaneous penetration in the patient's skin.

76. The method of claim 75, wherein the steps of forming, positioning and expanding are performed through a cannula positioned within the percutaneous penetration.

77. The method of claim 73, further comprising anchoring the expandable member to one or both of the spinous processes.

78. An interspinous device for stabilizing at least one spinal motion segment comprising a first vertebra having a first spinous process and a second vertebra having a second spinous process, the device comprising:
an axial dimension and a radial dimension substantially transverse to the axial dimension, wherein the axial dimension is greater when the device is in an undeployed configuration and the radially dimension is greater when the device is in a deployed configuration.

79. The interspinous device of claim 78, wherein the axial dimension is defined by a central member and the radial dimension is defined at least in part by a plurality of radially expandable members.

80. The interspinous device of claim 79, comprising a first strap extending between a first pair of the radially expandable members and a second strap extending between a second pair of the radially expandable members.

81. The interspinous device of claim 79, wherein the radially expandable members comprise linkages.

82. The interspinous device of claim 79, wherein the radially expandable members comprise deformable struts.

83. The interspinous device of claim 79, wherein the radially expandable members comprise a compressible material.

84. A system for stabilizing at least one spinal motion segment comprising a first vertebra having a first spinous process and a second vertebra having a second spinous process and an interspinous space defined between the first and second spinous processes, the system comprising:
the interspinous device of claim 78; and
a device for delivering the interspinous device in the undeployed state within the interspinous processes and for axially reducing and radially expanding the device from the undeployed state to the deployed state.

85. The system of claim 84, wherein the interspinous device is configured for delivery by the delivery device through a midline incision.

86. A method for stabilizing a vertebrae relative to another vertebrae wherein the vertebrae define an interspinous space, the method comprising:
introducing the interspinous device of claim 78 within the interspinous space when in the undeployed state;
and radially expanding and axially shortening the device to engage with the spinous processes.

87. The method of claim 86, further comprising forming an incision along the midline above the interspinous space, wherein the introducing the interspinous device comprises inserting the device within the midline incision.

88. An interspinous device for stabilizing at least one spinal motion segment comprising a first vertebra having a first spinous process and a second vertebra having a second spinous process, the device comprising:
a tubular member having a length and a diameter and being deployable from a first configuration to a second configuration, wherein one of the length and the diameter of the tubular member in the first configuration is changed when the tubular member is in the second configuration.

89. The device of claim 88 wherein the length of the tubular member in the first configuration is greater than the length of the tubular member in the second configuration.

90. The device of claim 88 wherein the diameter of the tubular member in the second configuration is greater than the diameter of the tubular member in the first configuration.

91. The device of claim 88 further comprising a retaining member circumferentially positioned about a portion of the length of the tubular member, wherein the diameter of the retained portion of the tubular member is less than the diameter of the unretained portion of the tubular member when the tubular member is in the second configuration.

92. The device of claim 91 wherein the diameter of the retained portion of the tubular member in the second configuration is sufficient to distract the first and second vertebrae relative to each other when the device is operably implanted between the first and second spinous processes.

93. The device of claim 91, wherein the retaining member is positioned about a central portion of the tubular member.

94. The device of claim 88, further comprising a core member positionable within the tubular member; and first and second hubs configured for coupling with a first end of the core member and a second end of the core member, respectively.

95. The device of claim 94, wherein the at least one of the hubs is configured to translate axially over the core member.

96. The device of claim 94, wherein the core member comprises segments of length which are removable from the core member.

97. The device of claim 88, wherein the tubular member comprises a polymer material.

98. The device of claim 88, wherein the retaining member comprises a braided or mesh material.

99. The device of claim 88, wherein a luminal surface of the tubular member is contoured.

100. A system for stabilizing at least one spinal motion segment comprising a first vertebra having a first spinous process and a second vertebra having a second spinous process, the system comprising:
the interspinous device of claim 88; and
a device for delivering the interspinous device when in the first configuration between the first and second spinous process and for deploying the interspinous device in the second configuration.

101. The system of claim 98 wherein the delivery device comprises:
an elongated shaft having a detachable distal end configured for insertion into the tubular member of the interspinous device; and
a handle mechanism for moving the elongated shaft wherein the movement causes the tubular member to be deployed from the first configuration to the second configuration.

102. A method for stabilizing a vertebrae relative to another vertebrae wherein the vertebrae define an interspinous space, the method comprising:
introducing the interspinous device of claim 88 within the interspinous space when in the first configuration; and
deploying the interspinous device to the second configuration.

103. A device for stabilizing at least one spinal motion segment comprising a first vertebra having a first spinous process and a second vertebra having a second spinous process, the device comprising:
an undeployed configuration having an axial dimension and a radial dimension substantially transverse to the axial dimension; and a deployed configuration having an axial dimension and a radial dimension substantially transverse to the axial dimension;
wherein the radial dimension of the undeployed configuration is less than the radial dimension in the deployed configuration.

104. The device of claim 103, wherein the axial dimension of the undeployed configuration is greater than the axial dimension in the deployed configuration.

105. The interspinous device of claim 103, wherein the radial dimension is defined at least in part by a plurality of radially expanding members.

106. The device of claim 105, wherein the radially expanding members comprise deformable struts.

107. The device of claim 105, wherein the radially expanding members comprise linkages.

108. The device of claim 105, comprising a first bracket extending between a first pair of the radially expandable members and a second bracket extending between a second pair of the radially expandable members.

109. The device of claim 107, wherein the brackets each comprise a substantially rigid central portion and two substantially flexible lateral portions.

110. The device of claim 103, wherein the device in the undeployed has a cylindrical shape.

111. The device of claim 103, wherein the device in the deployed has a “X” shape.

112. A system for stabilizing at least one spinal motion segment comprising a first vertebra having a first spinous process and a second vertebra having a second spinous process and an interspinous space defined between the first and second spinous processes, the system comprising:
the device of claim 103; and
a device for delivering the device in the undeployed configuration within the interspinous processes and for radially expanding the device from the undeployed configuration to the deployed configuration.

113. The system of claim 112, wherein the device is configured for delivery by the delivery device through a midline incision.

114. A method for stabilizing a vertebra relative to another vertebra wherein the vertebrae define an interspinous space therebetween, the method comprising:
introducing the device of claim 103 within the interspinous space when in the undeployed configuration; and
radially expanding the device to selectively distract the spinous processes.

115. The method of claim 114, further comprising forming an incision along the midline above the interspinous space, wherein the introducing the interspinous device comprises inserting the device within the midline incision.

116. A posterior element distraction system for implantation at a spinal motion segment comprising a superior vertebra, an inferior vertebra, each vertebra comprising a posterior element comprising a spinous process, laminal portions and a set of facet joints, and further comprising an interspinous space between the spinous processes, the system comprising:
at least one lateral member for positioning on a side of the spinal motion segment and outside the interspinous space, wherein the at least one lateral member has an unexpanded configuration and an expanded configuration; and
first and second transverse members extending transversely from the at least one lateral member, wherein when the system is operatively implanted at a spinal motion segment and the at least one lateral member is in an expanded configuration, the transverse members are caused to contact a portion of either the superior or inferior posterior elements thereby providing distraction between the superior and inferior posterior elements.

117. The system of claim 116, wherein the system comprises two lateral members for positioning on opposite sides of spinal motion segment.

118. The system of claim 117, wherein each transverse member is a strap extending between the two lateral members.

119. The system of claim 117, further comprising a meshing about the two lateral members.

120. The system of claim 116, wherein the system comprises only one lateral member.

121. The system of claim 116, wherein the at least one lateral member comprises a balloon configuration.

122. The system of claim 117, wherein the at least one lateral member comprises a strut configuration.

123. The system of claim 118, wherein the at least one lateral member comprises a balloon and a strut.

124. The system of claim 116, wherein the transverse members have a pre-formed configuration.

125. The system of claim 116, wherein the transverse members have a flexible configuration.

126. A system for implanting the system of claim 116, the implantation system comprising:
a temporary distraction device having an expanded configuration and an unexpanded configuration and further configured for insertion within the interspinous space, wherein upon expansion of the device, the superior and inferior vertebrae are distracted from each other.

127. The system of claim 126, further comprising:
a working channel for delivering the temporary distraction device and the posterior distraction system, wherein the temporary distraction device is deliverable in an unexpanded configuration.

128. The system of claim 127, wherein the temporary distraction device and the posterior distraction system are deliverable simultaneously to the spinal motion segment.

129. The system of claim 126, wherein the temporary distraction device comprises a balloon.

130. A method for distracting at least a portion of a spinal motion segment comprising a superior vertebra, an inferior vertebra, each vertebra comprising a posterior element comprising a spinous process, laminal portions and a set of facet joints, and further comprising an interspinous space between the spinous processes, the method comprising:
inserting an expandable member laterally of the spinal motion segment; and
expanding the expandable member thereby distracting the superior vertebra and the inferior vertebra relative to each other.

131. The method of claim 130, further comprising: inserting two transverse members within the interspinous space.

132. The method of claim 130, wherein the distraction between the vertebrae is along the longitudinal axis of the spine.

133. The method of claim 130, wherein the distraction between the vertebrae is rotational.

134. A method for distracting at least a portion of a spinal motion segment comprising a superior vertebra, an inferior vertebra, each vertebra comprising a posterior element comprising a spinous process, laminal portions and a set of facet joints, and further comprising an interspinous space between the spinous processes, the method comprising:
distracting the superior vertebra and the inferior vertebra relative to each other until a desired amount of distraction is achieved;
inserting an implantable expandable member within the interspinous space; and
expanding the implantable expandable member to contact the spinous processes thereby maintaining the distraction achieved.

135. The method of claim 134, wherein the distracting comprises using another expandable member positionable within the interspinous space; and wherein the other expandable member is removed subsequent to the expanding of the implantable expandable.
